(19) United States
(12) Patent Application Publication
(10) Pub. No.: US 2017/0172543 A1
(43) Pub. Date: Jun. 22, 2017

(54) ULTRASOUND TRANSDUCER ARRAY

(71) Applicant: OLYMPUS CORPORATION, Tokyo (JP)

(72) Inventor: Sunao SATO, Yamato-shi (JP)

(73) Assignee: OLYMPUS CORPORATION, Tokyo (JP)

(21) Appl. No.: 15/452,784

(22) Filed: Mar. 8, 2017

Related U.S. Application Data
(63) Continuation of application No. PCT/JP2015/062373, filed on Apr. 23, 2015.

(30) Foreign Application Priority Data
Sep. 9, 2014 (JP) 2014-183512

(51) Int. Cl. A61B 8/00; G01S 7/521; A61B 8/12; A61B 1/018; A61B 1/06

(52) U.S. Cl. CPC A61B 8/4483; A61B 1/018; A61B 1/06; A61B 8/4272; A61B 8/12; G01S 7/521

(57) ABSTRACT

An ultrasound transducer array is formed by stacking an acoustic lens on a laminated body of a transducer section including transducers and first acoustic matching layers, and a second acoustic matching layer. Arrangement of the laminated body is such that a groove width of a groove portion of the transducer section is equal to or greater than a groove width of a groove portion of the second acoustic matching layer.

### CROSS REFERENCE TO RELATED APPLICATION

This application is a continuation application of PCT/JP2015/062373 filed on Apr. 23, 2015 and claims benefit of Japanese Application No. 2014-183512 filed in Japan on Sep. 9, 2014, the entire contents of which are incorporated herein by this reference.

### BACKGROUND OF THE INVENTION

1. Field of the Invention

The present invention relates to an ultrasound transducer array provided with an acoustic matching layer of a laminated structure.

2. Description of the Related Art

As disclosed in Japanese Patent Application Laid-Open Publication No. 2006-94981, for example, an ultrasound transducer array used for an ultrasound endoscope has a plurality of transducers and first acoustic matching layers which are segmented into strips and stacked on a sheet-shaped second acoustic matching layer, and an acoustic lens is formed on the surface of the second acoustic matching layer after bending the laminated body.

### SUMMARY OF THE INVENTION

An ultrasound transducer array according to an aspect of the present invention includes a plurality of transducers configured to ultrasonically vibrate, and to emit ultrasound, first acoustic matching layers arranged on the plurality of transducers, respectively, along a first direction that is a direction of emission of the ultrasound, and a second acoustic matching layer stacked on the first acoustic matching layers, along the first direction, where the second acoustic matching layer includes a main body portion configured to position the transducers and the first acoustic matching layers at a first interval that is a predetermined interval, and a plurality of tooth portions formed of a same material as the main body portion, the plurality of tooth portions being a plurality of protrusions provided to the main body portion at positions facing surfaces, of the first acoustic matching layers, on an ultrasound emission direction side, the plurality of protrusions having a width that is equal to or greater than a width of the first acoustic matching layers and arranged at a second interval of a width that is equal to or smaller than the first interval.

### BRIEF DESCRIPTION OF THE DRAWINGS

FIG. 1 is a configuration diagram of an entire ultrasound endoscope according to a first embodiment of the present invention;

FIG. 2 is an explanatory diagram showing a distal end portion of the endoscope according to the first embodiment of the present invention;

FIG. 3 is a cross-sectional diagram showing an ultrasound transducer array in a nosepiece according to the first embodiment of the present invention;

FIG. 4 is an explanatory diagram showing transducers and an acoustic matching layer before bending according to the first embodiment of the present invention;

FIG. 5 is an explanatory diagram showing the transducers and the acoustic matching layer after bending according to the first embodiment of the present invention;

FIG. 6 is an explanatory diagram, according to the first embodiment of the present invention, showing an example 1 of a case where the transducers and the acoustic matching layer do not face each other;

FIG. 7 is an explanatory diagram, according to the first embodiment of the present invention, showing an example 2 of a case where the transducers and the acoustic matching layer do not face each other;

FIG. 8 is an explanatory diagram showing transducers and acoustic matching layer according to a second embodiment of the present invention; and

FIG. 9 is an explanatory diagram showing the transducers and the acoustic matching layer after bending according to the second embodiment of the present invention.

### DETAILED DESCRIPTION OF THE PREFERRED EMBODIMENTS

Hereinafter, embodiments of the present invention will be described with reference to the drawings. Note that the diagrams are schematic, and the relationship between a thickness and a width of each member, the ratio of thicknesses of members and the like are not actual, and it is needless to say that the relationship between dimensions and the ratios may be different between the drawings.

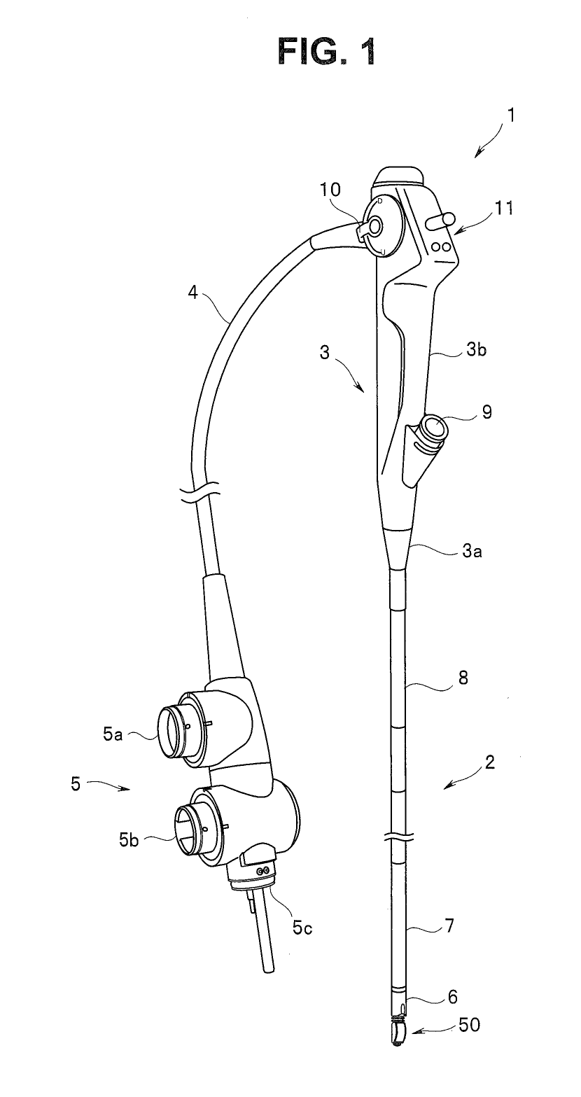

First, a first embodiment of the present invention will be described. FIG. 1 shows an ultrasound endoscope 1 provided with an ultrasound transducer array of the present invention, the ultrasound endoscope 1 being an electronic scanning ultrasound endoscope including an ultrasound transducer unit 50 on a distal end side of an insertion section 2 which is formed to have an elongated tubular shape and which is to be inserted into a body cavity or the like. An operation section 3, which serves also as a grasping portion, is continuously provided on a proximal end side of the insertion section 2 of the ultrasound endoscope 1, and a connector section 5 is arranged on a distal end side of a universal cord 4 extending from a side portion of the operation section 3.

The insertion section 2 is configured by including a rigid portion 6 which is provided continuously to the ultrasound transducer unit 50 on the distal end side, a bending portion 7 which is continuously provided on a rear end side of the rigid portion 6 and which is configured to be able to bend in an up-down direction, for example, and a flexible tube portion 8 which is continuously provided on a rear end side of the bending portion 7. The flexible tube portion 8 is a long tubular member with a small diameter which is provided between the bending portion 7 and the operation section 3 and which is formed to be flexible so as to be passively flexed.

The operation section 3 includes a bend preventing portion 3a which is connected to the flexible tube portion 8 while covering a proximal end of the flexible tube portion 8, and a grasping portion 3b which is provided continuously to the bend preventing portion 3a and which is to be grasped by the hand of a user at the time of use of the endoscope 1. Various operation members are arranged on an upper end side of the grasping portion 3b, and a treatment instrument insertion opening 9 for guiding a treatment instrument into a body cavity is provided on a lower end side of the grasping portion 3b, at a position above the bend preventing portion 3a, for example. The operation members provided to the operation section 3 include a bending lever 10 for performing a bending operation of the bending portion 7, a plurality of operation buttons 11 for performing an air/water feeding operation or a suction operation, operations related to image pickup and illumination, and the like.

The universal cord 4 is a composite cable allowing insertion of various signal lines and the like which reach the operation section 3 from the distal end of the insertion section 2, through the bending portion 7 and the flexible tube portion 8, and which extend from the operation section 3, insertion of a light guide of a light source device (not shown), and insertion of an air/water feeding tube extending from an air/water feeding device (not shown). The connector section 5 arranged on a distal end side of the universal cord 4 is configured by including an ultrasound connector 5a which is to be connected to an ultrasound observation device (not shown), an electrical connector portion 5b which is to be connected to various signal cables, and a light source side connector 5c which is to be connected to the light source device or the air/water feeding device (not shown).

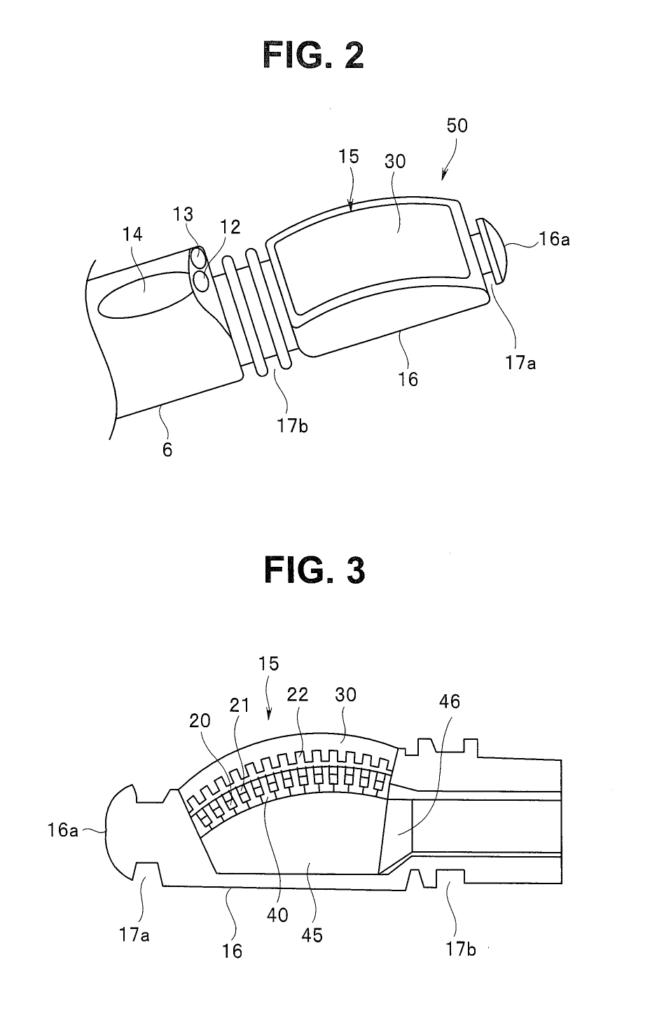

Next, a configuration of the distal end side of the insertion section 2 will be described with reference to FIG. 2. As shown in FIG. 2, the rigid portion 6 on the distal end side of the insertion section 2 is provided with an objective lens window 12 configuring an observation optical system, an illumination lens window 13 configuring an illumination optical system, and a treatment instrument outlet opening 14 through which a treatment instrument such as a puncture needle is to be guided out, for example.

The ultrasound transducer unit 50, which is provided continuously to the rigid portion 6, is configured by including an ultrasound transducer array 15, and a nosepiece 16 for housing the ultrasound transducer array 15. The ultrasound transducer array 15 includes an acoustic lens 30 which is integrally disposed and held in a housing portion, which is a recessed portion formed at a substantially center portion of the nosepiece 16, the acoustic lens 30 forming an ultrasound transmission/reception surface along a longitudinal direction of the insertion section 2.

Moreover, a substantially-cylindrical protruding portion 16a is provided to a distal end of the nosepiece 16, and a first balloon retention groove 17a is formed on an outer circumference of the protruding portion 16a, on a proximal portion side, and a second balloon retention groove 17b is formed on an outer circumference of a coupling portion between the rigid portion 6 and the nosepiece 16. A thin, contractible balloon formed of silicone rubber or latex rubber, for example, is detachably interposed between the first balloon retention groove 17a and the second balloon retention groove 17b while covering the nosepiece 16.

As shown in FIG. 3, the ultrasound transducer array 15 includes a plurality of transducers 20 which are arranged in a curved shape along a convex surface, and the plurality of transducers 20 are electrically connected to a wiring substrate 45 housed inside the nosepiece 16. A plurality of signal cables 46 forming a signal line and a ground line extend from the wiring substrate 45, and the signal cables 46 are connected to the ultrasound connector 5a through the insertion section 2.

Note that a piezoelectric element sandwiching a known piezoelectric device between an upper electrode and a lower electrode, or a capacitive element having a gap between an upper electrode and a lower electrode which are separated by a column by a predetermined distance may be applied as the transducer 20, for example. Also, a backing material 40 is arranged on a back side of a lower electrode of the transducer 20 so as to attenuate unnecessary ultrasound. As the backing material 40, a base material of an insulating material such as epoxy resin, silicone, urethane or various types of elastomer with which a filler material such as aluminum oxide, zirconia or titanium oxide is mixed may be used, for example.

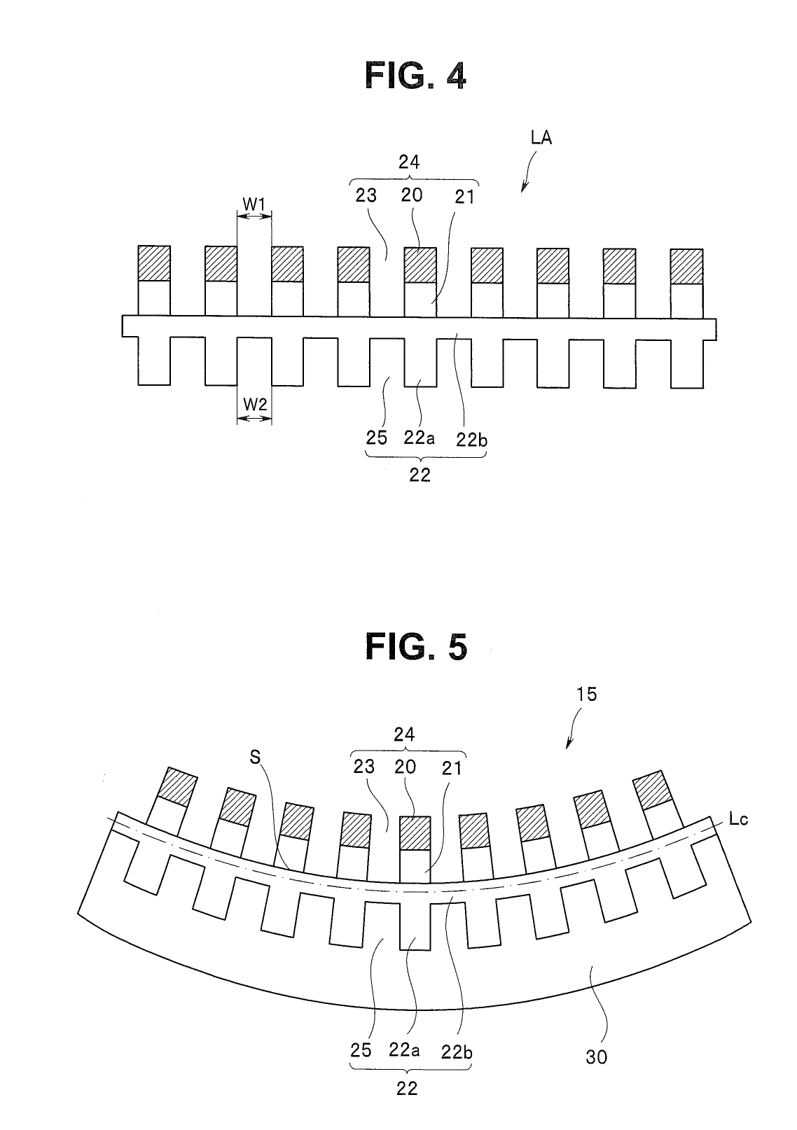

As shown in FIGS. 3 and 4, the ultrasound transducer array 15 includes, on a back side of the acoustic lens 30 held at the substantially center portion of the nosepiece 16, a second acoustic matching layer 22, first acoustic matching layers 21, and a plurality of transducers 20, and the shape and the material of the ultrasound transducer array 15 are such that acoustic impedance from the transducers 20 to a living body can be gradually reduced and desirable ultrasound propagation efficiency can be achieved.

More specifically, the transducers 20 and the first acoustic matching layers 21 are formed as a transducer section 24 including a plurality of first groove portions 23, by segmenting a thin-plate structure integrally bonding the transducers 20, the first acoustic matching layers 21, and the second acoustic matching layer 22 into strips by dicing. Also, the second acoustic matching layer 22 is foamed to have a comb shape including a plurality of tooth portions 22a protruding toward the acoustic lens 30, and a main body portion 22b which holds the plurality of tooth portions 22a and which is in contact with the first acoustic matching layers 21.

Second groove portions 25 arranged facing the first groove portions 23 of the first acoustic matching layers 21 are formed between the plurality of tooth portions 22a of the second acoustic matching layer 22. Due to the effect of the shape combining the plurality of tooth portions 22a and the second groove portions 25, the acoustic impedance from the first acoustic matching layers 21 to the second acoustic matching layer 22 may be changed smoothly, and the speed of the ultrasound propagated from the transducers 20 may be made close to desired speed. As a result, desirable acoustic impedance matching may be realized, and the ultrasound transmission efficiency may be increased and the sensitivity may be increased.

Furthermore, according to the ultrasound transducer array 15 of the present embodiment, acoustic matching layers which are capable of achieving desired acoustic impedance are provided and the ultrasound propagation efficiency may be increased, and also, the yield of manufacturing of the transducer array, including a bending process, may be maintained.

Therefore, the groove width of the second groove portions 25 of the second acoustic matching layer 22 is set to be equal to or less than the groove width of the first groove portions 23 of the transducer section 24, and the second acoustic matching layer 22 is arranged with respect to the first acoustic matching layers 21 in such a way that a second groove portion 25 faces a first groove portion 23 and fits within the groove width of the first groove portion 23. As shown in FIG. 4, in the present embodiment, a groove width W1 of the first groove portion 23 and a groove width W2 of the second groove portion 25 are set equal to each other (W1=W2), and a center of the groove width of the first groove portion 23 and a center of the groove width of the second groove portion 25 are on a same line.

As shown in FIG. 5, a laminated body LA of the transducer section 24 including such first groove portions 23 and the second acoustic matching layer 22 including such second groove portions 25 are bent along a convex surface, and the acoustic lens 30 is further stacked to cover the tooth portions 22a of the second acoustic matching layer 22, and the ultrasound transducer array 15 is thereby formed. The acoustic lens 30 is stacked in such a way as to cover the tooth portions 22a while filling the second groove portions 25 of the second acoustic matching layer 22. Note that the acoustic lens 30 may be stacked after filling the second groove portions 25 with a member of a different material from the acoustic lens 30.

In such a case, the laminated body LA of the transducer section 24 and the second acoustic matching layer 22 has a laminated structure with high tolerance against a mechanical stress which is caused by bending. That is, the groove widths and the positional relationship of the first groove portions 23 and the second groove portions 25 allow the laminated body LA to be a laminated structure according to which an inconvenience such as peeling of the transducer section 24 and the second acoustic matching layer 22 is not caused at the time of a bending process, and thus, a product with high ultrasound propagation efficiency may be obtained without reducing the manufacturing yield.

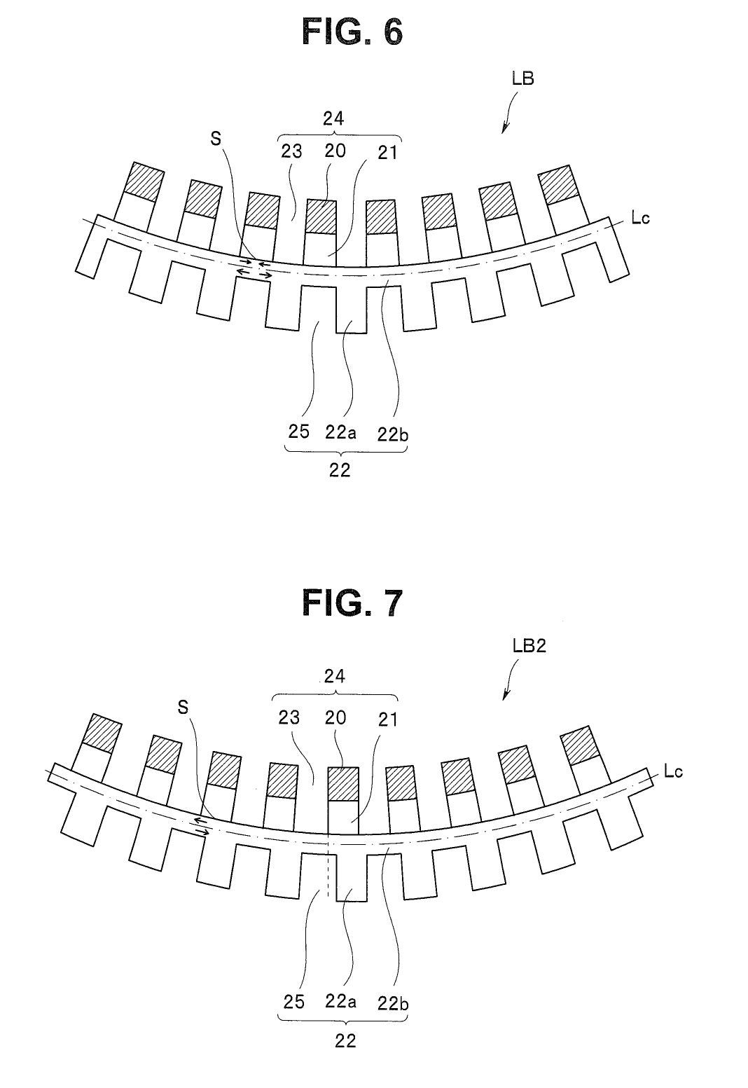

A laminated body LB will now be described, as shown in FIG. 6, for example, as a comparative example of the laminated body LA of the present embodiment, the laminated body LB having a laminated structure according to which the first groove portions 23 of the transducer section 24 and the second groove portions 25 of the second acoustic matching layer 22 are not arranged facing each other, but the tooth portions 22a of the second acoustic matching layer 22 are arranged facing the positions of the first groove portions 23 of the transducer section 24.

According to the laminated body LB of such an arrangement, when the main body portion 22b of the second acoustic matching layer 22 is bent at a neutral surface Lc at the time of the bending process, the bottom side of the second groove portions 25 is extended, and the corresponding first acoustic matching layer 21 side is compressed in the curvature radius direction of bending as shown by arrows in FIG. 6. Accordingly, due to the stress applied to a bonding interface S between the main body portion 22b and the first acoustic matching layers 21, interfacial peeling is highly likely to occur between the main body portion 22b and the first acoustic matching layers 21. Moreover, the interfacial peeling may further proceed due to exposure to cleaning/disinfection chemicals, sterilizing gas or the like.

Furthermore, as shown in FIG. 7, in the case of a laminated body LB2 according to which the first groove portions 23 of the first acoustic matching layers 21 and the second groove portions 25 of the second acoustic matching layer 22 substantially face each other but are shifted as shown by a broken line in the drawing, not only is the possibility of occurrence of interfacial peeling high, but a desirable bent shape is hard to realize.

That is, according to the laminated body LB2, interfacial peeling is highly likely to occur between the main body portion 22b and the first acoustic matching layers 21 due to the stress applied to the bonding interface S corresponding to the bottom side of the second groove portions 25 as shown by arrows in FIG. 7, and also, the first groove portions 23 and the second groove portions 25 are shifted from each other, and thus, variance in the tendency of the bottom side of the second groove portions 25 to deform becomes great. Accordingly, a desirable bent shape is hard to realize, and resolution is reduced due to uneven gap between the transducers 20 of the transducer section 24.

Note that also in a case where the first groove portions 23 of the first acoustic matching layers 21 and the second groove portions 25 of the second acoustic matching layer 22 face each other and centers of the groove widths of the first groove portions 23 and the second groove portions 25 coincide with each other, if the groove width of the first groove portions 23 is smaller than the groove width of the second groove portions 25, interfacial peeling is highly likely to occur due to the stress generated between the main body portion 22b and the first acoustic matching layers 21.

On the other hand, with the laminated body LA according to the present embodiment, the second groove portions 25 do not overlap the bonding interface S between the main body portion 22b and the first acoustic matching layers 21 in the curvature radius direction of bending when the main body portion 22b of the second acoustic matching layer 22 is bent at the neutral surface Lc, and thus, a stress applied to the bonding surface S between the main body portion 22b of the second acoustic matching layer 22 and the first acoustic matching layers 21 is small, and interfacial peeling is not caused between the main body portion 22b of the second acoustic matching layer 22 and the first acoustic matching layers 21. Therefore, a product with high ultrasound propagation efficiency may be obtained without reducing the manufacturing yield.

According to the ultrasound transducer array 15 having such a laminated body LA, the most suitable materials for forming the first acoustic matching layers 21, the second acoustic matching layer 22, and the acoustic lens 30 may be selected with a relatively high degree of freedom. For example, the first acoustic matching layers 21 may be formed of epoxy resin, and the second acoustic matching layer 22 may be formed of engineering plastic, which is excellent in heat resistance, mechanical strength, and chemical resistance, but according to which impedance matching is difficult. As the engineering plastic, polyimide (PI), poly ether imide (PEI), polysulfone (PSF), or poly ether ether ketone (PEEK) may be used, for example.

Moreover, the acoustic lens 30 may maintain sufficient adherence strength due to anchoring effect while being formed of silicone rubber excellent in chemical resistance, by being stacked while filling the second groove portions 25 of the second acoustic matching layer 22.

As described above, according to the present embodiment, because the second acoustic matching layer 22, which is stacked on the first acoustic matching layers 21 of the transducer section 24, is formed to have a comb shape including a plurality of tooth portions 22a which are arranged while being separated by the second groove portions 25, and the main body portion 22b holding the plurality of tooth portions 22a, the acoustic impedance may be desirably matched due to the effect of the shapes of the plurality of tooth portions 22a and the second groove portions 25. Accordingly, the ultrasound transmission efficiency may be increased, and the sensitivity may be increased.

Furthermore, by making the groove width of the second groove portions 25 of the second acoustic matching layer 22 equal to or less than the groove width of the first groove portions 23 of the transducer section 24, and arranging the second groove portions 25 to face the first groove portions 23 and to be within the groove width of the first groove portions 23, a great stress which would cause peeling at the bonding interface between the first acoustic matching layers 21 and the second acoustic matching layer 22 may be prevented from being applied at the time of the bending process. Accordingly, in cooperation with the effect of the shape of the acoustic matching layer, enhancement in the ultrasound performance and an increase in the yield of production may be achieved at the same time.

Moreover, by arranging the first groove portions 23 and the second groove portions 25 to face each other, an evenly bent shape may be achieved, and thus, the transducers 20 may be evenly arranged. Accordingly, ultrasound may be emitted with evenly spaced ultrasound scanning lines, and a reduction in the resolution due to unevenness in the scanning lines may be prevented.

Furthermore, by filling the second groove portions 25 by the same material as the acoustic lens 30, a laminated structure according to which the acoustic impedance is gradually changed from the second acoustic matching layer 22 to the acoustic lens 30 may be obtained. Therefore, more desirable acoustic impedance matching may be realized, and the ultrasound transmission efficiency may be increased and the sensitivity may be increased.

Next, a second embodiment of the present invention will be described. In the second embodiment, the arrangement of the first groove portions 23 and the second groove portions 25 of the laminated body LA of the first embodiment is slightly changed to obtain a laminated body LA2.

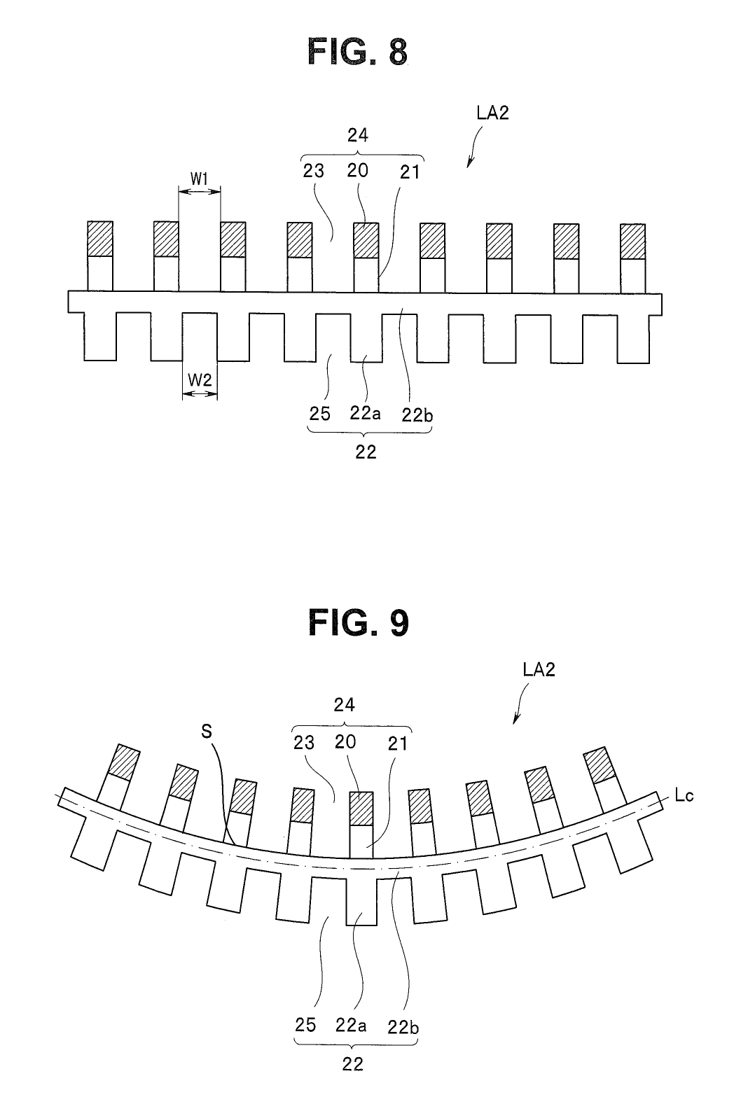

As shown in FIG. 8, according to the laminated body LA2 of the second embodiment, the groove width W1 of the first groove portion 23 is greater than the groove width W2 of the second groove portion 25 (W1>W2), and the first groove portion 23 and the second groove portion 25 are arranged facing each other with the second groove portion 25 positioned within the groove width of the first groove portion 23. Note that the first groove portion 23 and the second groove portion 25 are desirably arranged with the centers of the groove widths on the same line, but the center of the groove width of the first groove portion 23 and the center of the groove width of the second groove portion 25 do not necessarily have to coincide with each other.

As shown in FIG. 9, when such a laminated body LA2 is bent, the main body portion 22b of the second acoustic matching layer 22 is extended on the bottom side of the second groove portions 25 and compressed on the bottom side of the first groove portions 23 with the neutral surface Lc as the boundary. At this time, because the groove width of the first groove portion 23 facing the second groove portion 25 is greater than the second groove portion 25, even if a relatively great force in the extending direction is applied to the bottom side of the second groove portion 25, a great force which would cause interfacial peeling is not applied to the bonding interface S between the first acoustic matching layers 21 of the transducer section 24 and the main body portion 22b of the second acoustic matching layer 22.

Also in the second embodiment, as in the first embodiment, a product with high ultrasound propagation efficiency may be obtained without reducing the manufacturing yield.

What is claimed is:

1. An ultrasound transducer array comprising:
a plurality of transducers configured to ultrasonically vibrate, and to emit ultrasound;
first acoustic matching layers arranged on the plurality of transducers, respectively, along a first direction that is a direction of emission of the ultrasound; and
a second acoustic matching layer stacked on the first acoustic matching layers, along the first direction,
wherein the second acoustic matching layer includes
a main body portion configured to position the transducers and the first acoustic matching layers at a first interval that is a predetermined interval, and
a plurality of tooth portions formed of a same material as the main body portion, the plurality of tooth portions being a plurality of protrusions provided to the main body portion at positions facing surfaces, of the first acoustic matching layers, on an ultrasound emission direction side, the plurality of protrusions having a width that is equal to or greater than a width of the first acoustic matching layers and arranged at a second interval of a width that is equal to or smaller than the first interval.

2. The ultrasound transducer array according to claim 1, wherein arrangement is such that a center of a width of the first interval and a center of a width of the second interval are on a same line.

3. The ultrasound transducer array according to claim 1, further comprising an acoustic lens that is stacked on the second acoustic matching layer in a manner filling the second interval and covering the tooth portions.

4. The ultrasound transducer array according to claim 1, wherein the second acoustic matching layer is formed of engineering plastic.
